(19) United States
(12) Patent Application Publication
(10) Pub. No.: US 2024/0115819 A1
(43) Pub. Date: Apr. 11, 2024

(54) In-Line Nasal Delivery Device

(71) Applicant: Impel Pharmaceuticals Inc., Seattle, WA (US)

(72) Inventors: John D. Hoekman, Seattle, WA (US); Christopher Fuller, Seattle, WA (US); Craig Kohring, Seattle, WA (US)

(21) Appl. No.: 18/538,917

(22) Filed: Dec. 13, 2023

Related U.S. Application Data
(63) Continuation of application No. 18/297,601, filed on Apr. 8, 2023, now abandoned.
Application No. 18/297,601 is a continuation of application No. 17/866,222, filed on Jul. 15, 2022, now abandoned.
Application No. 17/866,222 is a continuation of application No. 17/585,099, filed on Jan. 26, 2022, now abandoned.
Application No. 17/585,099 is a continuation of application No. 15/759,447, filed on Mar. 12, 2018, now Pat. No. 11,266,799.
(60) Provisional application No. 62/216,789, filed on Sep. 10, 2015.

(51) Int. Cl. A61M 15/08; A61M 11/02; A61M 15/00

(52) U.S. Cl. CPC A61M 15/08; A61M 11/02; A61M 15/009; A61M 2210/0618

(57) ABSTRACT

A delivery device for a compound including: a housing, vial holding a compound; and a source of propellant, wherein the housing provides an inlet and an outlet for the vial, wherein the inlet is in fluid communication with the source of propellant and is directed against the compound and the outlet allows for delivery of the compound.

### CROSS REFERENCE TO RELATED APPLICATIONS

This application is a continuation of U.S. patent application Ser. No. 18/297,601, filed Apr. 8, 2023, which is a continuation of Ser. No. 17/866,222, filed Jul. 15, 2022, which is a continuation of U.S. patent application Ser. No. 17/585,099, filed Jan. 26, 2022, which is a continuation of U.S. patent application Ser. No. 15/759,447 (now U.S. Pat. No. 11,266,799) which is a National Stage Entry of PCT/US2016/051169 filed Sep. 9, 2016, which claims priority to U.S. Provisional Patent Application No. 62/216,789 filed Sep. 10, 2015, the contents of all of which are hereby incorporated by reference herein in their entirety.

### BACKGROUND

Depositing drug on the olfactory region of the nasal cavity is difficult to accomplish due to the complex architecture of the nasal cavity and the turbinate guided air path for inhaled breath through the nose. These natural structures act to prevent materials from depositing on the olfactory region as a way to protect this entry way into the central nervous system (CNS). Nasal drop or spray devices, such as the Pfieffer nasal spray devices (Radolfzell, Germany), are designed to saturate the lower nasal cavity. Drug deposited on the lower nasal cavity is absorbed into the blood stream instead of the CNS, eliminating an advantage of using the nasal route for CNS delivery.

A more elegant approach to the intranasal delivery of compounds or mixtures is needed.

### SUMMARY

Shown and described is one implementation of a device for the intranasal delivery of a compound including a y-junction having a base, a first branch of the y-junction radiating from the base, a second branch of the y-junction radiating from the base, a third branch of the y-junction radiating from the base, and an internal dose loading channel of the y-junction, a metered dose pump in fluid communication with the first branch of the y-junction, a conical spring associated with the second branch of the y-junction, a dose chamber in fluid communication with the third branch of the y-junction, a nozzle associated with the dose chamber, a diffuser compression fit between the internal dose loading channel and the dose chamber, an actuator grip surrounding the y-junction, and a housing, the y-junction residing within the housing.

In one aspect, the in-line nasal delivery device further includes a propellant canister in fluid communication with the second branch of the y-junction and held by the actuator grip, the conical spring between the propellant canister and the second branch of the y-junction.

In another aspect, the in-line nasal delivery device further includes a vial in fluid communication with the metered dose pump.

In yet another aspect, the in-line nasal delivery device further includes a pump fitment securing the metered dose pump to the vial.

In another implementation, shown and described is an in-line nasal delivery device for the intranasal delivery of a compound including a housing, the housing including a tip, an actuator, and a dose chamber, the tip and the dose chamber in fluid communication within the housing, a nozzle at a distal portion of the tip, the nozzle providing an outlet for the compound, and a pump in fluid communication with the dose chamber, the pump to move the compound into the dose chamber.

In one aspect, the in-line nasal delivery device further includes a propellant canister in communication with the housing, the propellant canister having a propellant valve and in fluid communication with the dose chamber.

In another aspect, the in-line nasal delivery device further includes a vial of compound cooperative with the pump to move the compound into the dose chamber.

In another aspect, the in-line nasal delivery device when actuated compresses the pump moving the compound into the dose chamber and actuation of the propellant valve disperses the propellant pushing the compound providing for the compound to exit the device through the nozzle openings.

The invention will best be understood by reference to the following detailed description of various implementations, taken in conjunction with any accompanying drawings. The discussion below is descriptive, illustrative and exemplary and is not to be taken as limiting the scope defined by any appended claims.

### DESCRIPTION OF THE DRAWINGS

The foregoing aspects and many of the advantages will be more readily appreciated as the same become better understood by reference to the following detailed description, when taken in conjunction with the accompanying drawings, wherein:

FIG. 1 shows a cross section of the in-line nasal delivery device.

FIG. 2 shows a cross section of the in-line nasal delivery device in the stages of rest and actuation. FIG. 2A shows the in-line nasal delivery device at rest with FIG. 2B showing the actuation of the pump and FIG. 2C showing actuation of the propellant valve.

FIG. 3 shows a cross section of another implementation of the in-line nasal delivery device.

FIG. 4 shows a cross section of the diffuser as seated within the device.

FIG. 5A shows an exploded view of the dose chamber and the y-junction unassembled.

FIG. 5B shows an exploded view of the dose chamber and y-junction in cooperation.

FIG. 6 shows arrows representing both dose and propellant flow.

FIG. 7 shows the actuator grip and conical spring arrangement.

FIG. 8 shows a cross section of the optional nose cone and a side elevation of the optional nose cone.

### DETAILED DESCRIPTION

When trade names are used herein, applicants intend to independently include the trade name product and formulation, the generic compound, and the active pharmaceutical ingredient(s) of the trade name product.

For clarity of disclosure, and not by way of limitation, the detailed description is divided into the subsections which follow.

Unless defined otherwise, all technical and scientific terms used herein have the same meaning as commonly understood by one of ordinary skill in the art pertinent to the methods, apparatus and compositions described. As used herein, the following terms and phrases have the meanings ascribed to them unless specified otherwise:

“A” or “an” may mean one or more.

In one implementation, the in-line nasal delivery device 1 delivers compound into the nasal cavity and deposits compound in the nasal cavity beyond the nasal valve. The deposition includes the turbinates and/or the olfactory region. The compound delivered is a liquid. The compound may be a drug, active pharmaceutical ingredient, or a pharmaceutical formulation. The compound delivered may be a dose.

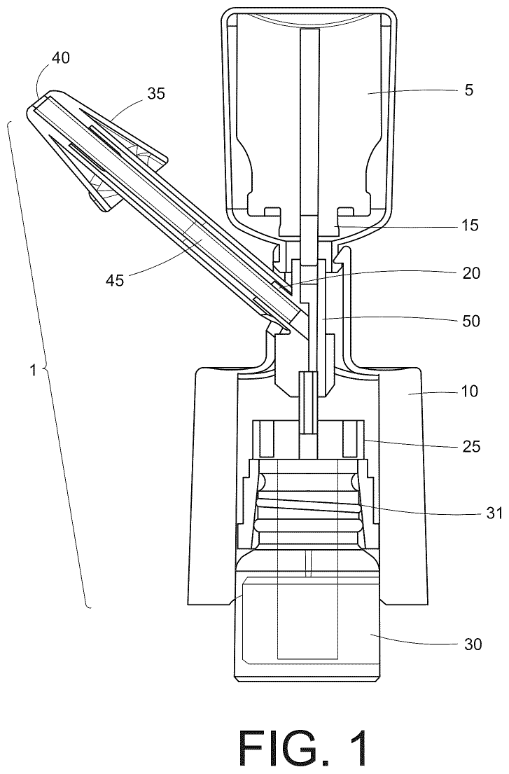

As shown in FIG. 1, the in-line nasal delivery device 1 includes a housing 10, diffuser 20, tip 35, nozzle 40, dose chamber 45, an actuator 50, and a pump 25 to move the compound into the dose chamber 45. In one aspect, the in-line nasal device 1 is associated and cooperative with a propellant canister 5, a propellant valve 15, and a vial 30 of compound cooperative with the pump 25 to move the compound into the dose chamber 45.

In one aspect, the diffuser 20 is a frit 21. The diffuser provides for the conversion of the liquefied propellant in the propellant canister 5 to gas and/or an increase in temperature of the propellant.

In one aspect, the propellant valve 15 is a metered dose propellant valve 16.

In one aspect, the compound is supplied in the form of a sealed vial 30, e.g., of glass, that contains a quantity of liquid. In one aspect, the vial 30 has a neck 31 that is sealed by a removable closure 32 (not shown), for example but not limited to sealed with a plastic cover, crimped metal seal, and rubber stopper (for stability and sterility purposes). In one aspect, the vial 30 may contain the active pharmaceutical ingredient. When the closure 32 is removed, the device 1 is engaged with the vial 30, in one aspect, by cooperation with the neck 31 of the vial 30. A pump 25 moves the compound into the dose chamber 45.

The propellant canister 5 is a canister of a compressed gas or a liquefied propellant. Compressed gases include but are not limited to compressed air and compressed hydrocarbons. In one aspect, nitrogen or carbon dioxide. Liquefied propellants include but are not limited to chlorofluorocarbons and hydrofluoroalkanes. The canister 5 will generally be provided with a propellant valve 15 by which the gas flow can be controlled.

The tip 35 includes a nozzle 40. In one aspect, the nozzle 40 has a plurality of nozzle openings 41 (not shown). Thru the plurality of nozzle openings 41, the compound and propellant is delivered to the nasal cavity.

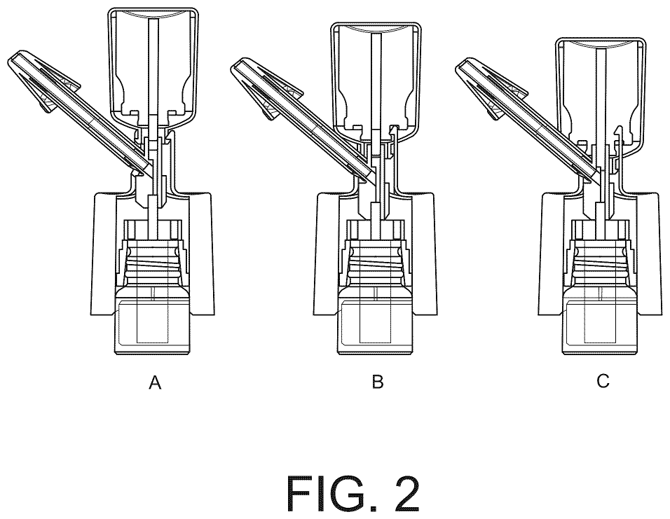

Actuation of the propellant canister 5 is effectively coordinated with actuation of the pump 25 for the vial 30 for the compound. The arrangement may be such that actuation of the vial 30 for the compound causes actuation of the propellant canister 5. FIG. 2 shows the device 1 at rest (FIG. 2A) and in actuation (FIGS. 2B and 2C).

As an example, the staging of the device 1 actuation is as follows. The housing 10 is compressed to prime the propellant canister 5. When the housing 10 is compressed, an actuator 50 remains stationary in the housing 10 while the propellant canister 5 and the vial 30 move towards the actuator 50. At this time, the propellant valve 15 associated with the propellant canister 5 is not actuated by compression. The actuator 50 acts upon the pump 25 compressing the pump 25 and the compound from the vial 30 is moved into the dose chamber 45. After a majority of the compound has moved into the dose chamber 45, the actuator 50 acts upon the propellant valve 15 and the propellant valve 15 begins to compress. The continued depression of the actuator 50 releases the propellant from the propellant canister 5. The propellant pushes the compound as it exits the device 1 through the nozzle openings 41 of the nozzle 40 located in the tip 35. The actuator 50 provides for first actuation of the pump 25, then once the pump 25 bottoms out, the continued depression of the actuator 50 provides for release of the propellant from the canister 5.

In an alternative implementation of the device 1 (not shown), the device 1 does not include a diffuser 20.

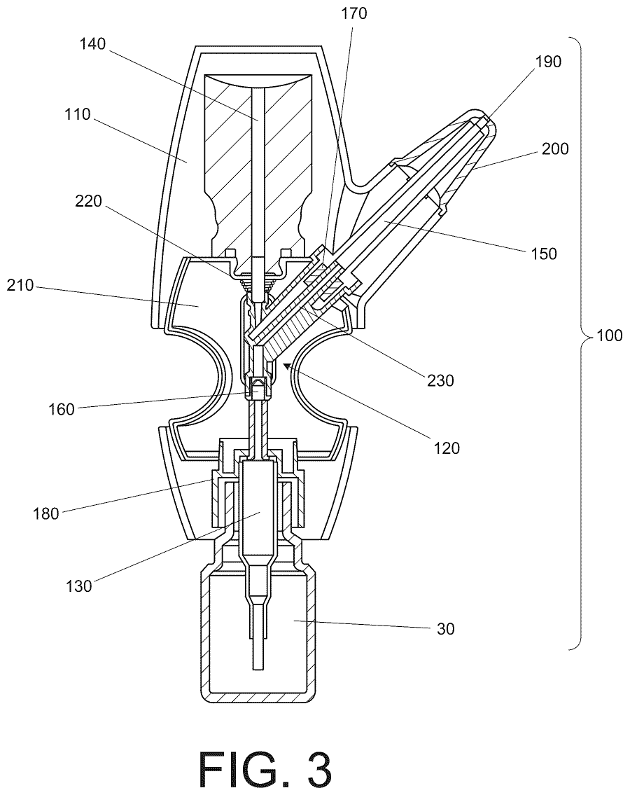

FIG. 3 shows yet another implementation of the device 100. The device 100 can deliver a single or multiple dose from a vial 30 or other container. The device 100 allows for multiple doses to be delivered from the vial 30, or a single dose. For example, the vial 30 may contain a volume of compound for multiple doses, while the user may decide to only deliver a single dose from the vial 30. The compound may be a drug, active pharmaceutical ingredient, or a pharmaceutical formulation.

Initially, the vial 30 may be separate from the rest of the assembled device 100. At the time of use, the device 100 and vial 30 are taken out of their respective packaging. Prior to use, the vial 30 will generally be sealed. In the aspect where the vial 30 is covered by a plastic cover, metal seal and stopper, the plastic cover and metal seal are pulled away from the top of the vial 30, and the rubber stopper is removed from the vial 30. The vial 30 may be screwed into a pump fitment 180 located at the base of the device 100. For example, but not limitation, the vial 30 may have female threads which can be screwed into male threads on a pump fitment 180, or vice versa. The vial 30 may contain, for example but not limited to, inclusive of end points, 2-3 ml, in another aspect 2-2.5 ml of compound.

As shown in FIG. 3, the device 100 includes a housing 110. The housing 110 contains components of the device 100 including the y-junction 120. The y-junction 120 has three branches radiating from a common base. The y-junction and its three branches may be a molded component. The y-junction 120 establishes both fluid and gas paths within the device 100, and connects the metered dose pump 130, the dose chamber 150, and the propellant canister 140 when the propellant canister 140 is assembled with the device.

As shown in FIG. 3, for use of the device 100, the user will generally orient the device 100 with the propellant canister 140 assembled and located at the top and the vial 30 assembled and located at the bottom. Housed within the device's 100 housing 110, the optional check-valve 160 (attached to the metered dose pump 130 stem) press fits into a receiving hub of a first branch of the y-junction 120. An internal bore provides fluid communication from the metered dose pump 130, through the optional check-valve 160 and y-junction 120, to the dose chamber 150. In one aspect, the check valve 160 is an elastomeric component that installs within a plastic housing between the metered dose pump 130 and the y-junction 120. The optional check valve 160: (a) reduces or eliminates dose leakage which could occur through the metered dose pump 130 if the pump stem was depressed and the propellant canister 140 was actuated; (b) allows for improved consistency in dose delivery by the device 100; and/or provides that compound is not pushed back down the internal dose loading channel 230 of the y-junction 120 and into the metered dose pump 130.

When oriented as to be used in operation, housed within the device's 100 housing 110, towards the top of the device 100, the propellant canister 140 press fits into a second branch of the y-junction 120, establishing the gas path through internal bores, through the diffuser 170 and to the dose chamber 150.

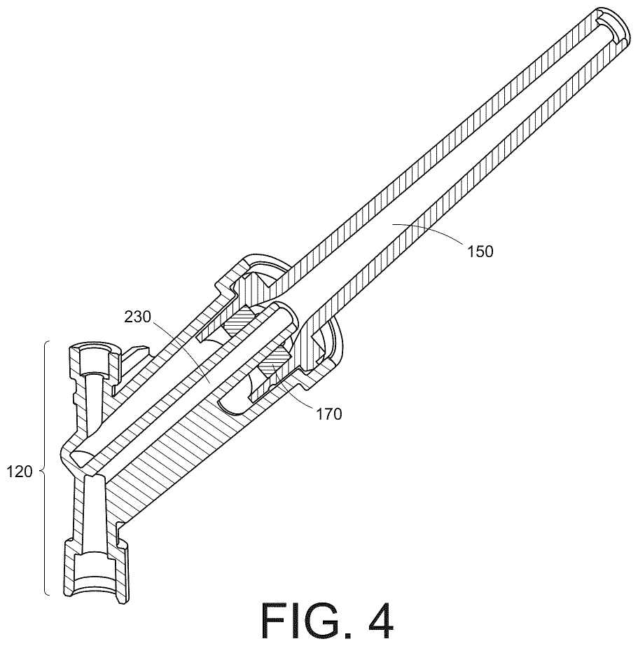

In this implementation of the device 100, the diffuser 170 is annular. As shown in FIG. 4, the annular diffuser 170 sits inside a bore on the back end of the dose chamber 150. The external diameter of the annual diffuser 170 is in a compression fit with the dose chamber 150. An internal dose loading channel 230 which is molded as a portion of the y-junction 120 fits into the inner bore of the annual diffuser 170 when the dose chamber 150 is installed onto the y-junction 120. The inner diameter of the annular diffuser 170 is in compression with the internal dose loading channel 230 portion of the y-junction 120. The annular diffuser 170 is seated between the outer wall of the internal dose loading channel 230 and the inner wall of the dose chamber 150, sealing against both of those surfaces to form the bottom of the dose chamber 150.

In one aspect, the diffuser 170 is a frit 171. The diffuser 170: (a) provides for the conversion of the liquefied propellant in the propellant canister 140 to gas; (b) provides an increase in temperature of the propellant; (c) acts to prevent the propellant from flowing back into the device 100; (d) acts to prevent the compound from flowing back into the device 100; and/or (e) acts to allows gas flow into the dose chamber 150 while preventing the compound from leaking out. The diffuser may be made of a porous polymer material.

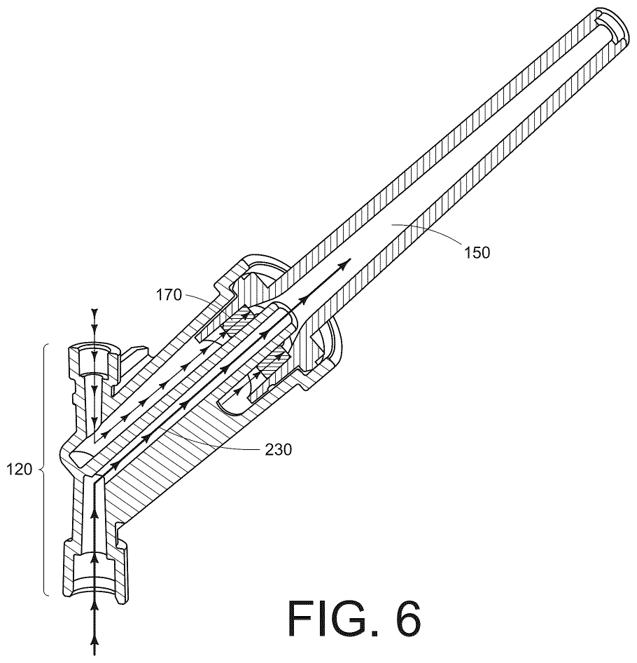

The relationship in operation of the device 100 between the compound, the annular diffuser 170, the inner dose loading tube 230, the dose chamber 150 and the y-junction 120 are shown at least in FIG. 6. In operation, the compound being loaded into the dose chamber 150 takes the less restrictive route, flowing out of the vial 30 and filling the dose chamber 150 rather than loading backwards through the annular diffuser 170 and into the delivery path of the propellant of the y-junction 120. In operation of the device 100, the staging of operation and the amount of time required for operation of the device allows the annular diffuser 170 to restrict compound from flowing back into the y-junction 120 for the period of time needed, as the propellant canister 140 is activated after compound loading. During proper device 100 use, the entire actuation of the device 100, including metered dose pump 130 and propellant canister 140, is approximately a second or less than a second. The loaded dose in the dose chamber 150 does not have enough time to flow backwards into the y-junction 120. Immediately after the dose chamber 150 is full, the propellant expels the compound from the device 100.

On the third leg of the y-junction 120 at a 45-degree angle, the dose chamber 150 press fits into the y-junction 120, completing the flow paths for both gas and fluid through the device. In one aspect, the angle is 30 degrees, 35 degrees, 40 degrees, 45 degrees, 50 degrees, 55 degrees, 60 degrees, inclusive of endpoints and intervening degrees.

The y-junction 120 may contain engagement ribs (not shown) to help secure and position the assembly within the housing 110 of the device 100.

The device 100 includes a pump fitment 180. The pump fitment 180 secures the metered dose pump 130 to the vial 30 and holds both components in place during device 100 use. One aspect of the pump fitment 180 is that it consists of engagement ribs that retain it within the housing 110, provide vertical displacement, and prevent rotation during installation of the vial 30.

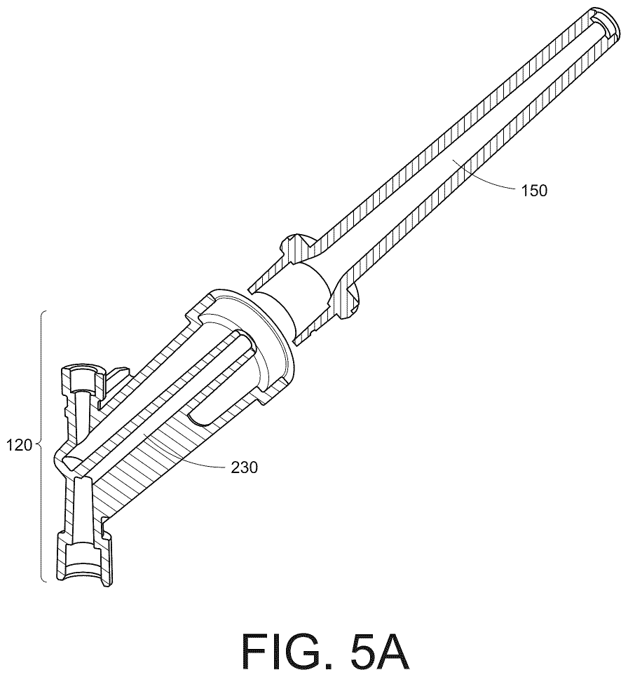

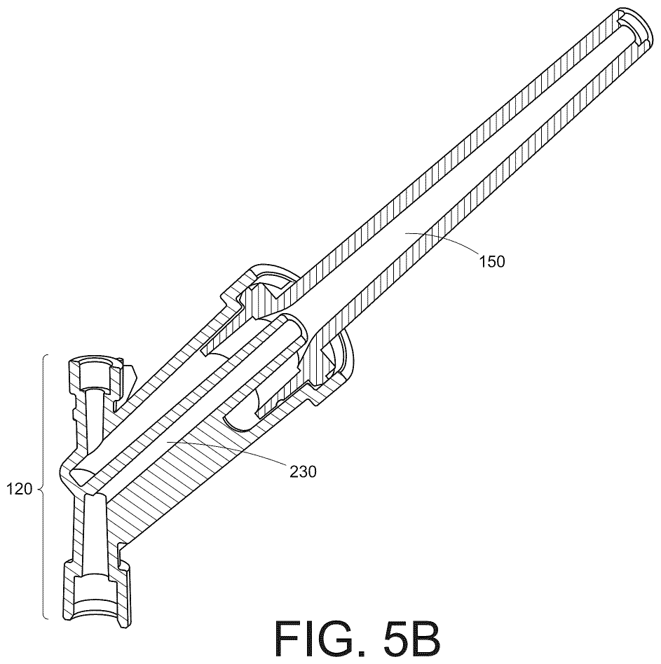

The device 100 includes a dose chamber 150. The dose chamber 150 receives and stores the compound that has been pushed out of the inner tube of the y-junction 120. When the propellant canister 140 is actuated, the y-junction 120 and dose chamber 150 are pressurized and the propellant gas expels the compound out of the dose chamber 150. As shown in FIGS. 5A and 5B, the dose chamber 150 is press fit into the y-junction 120. The nozzle 190 is installed into the end of the dose chamber 150 opposite where it is press fit into the y-junction 120.

The nozzle 190 is installed into the distal end (end opposite where the dose chamber 150 is press fit into the y-junction 120) of the dose chamber 150, forming a liquid and gas-tight seal around the outer diameter. During actuation of the device 100, propellant evacuates liquid compound from the dose chamber 150, pushing it out the nozzle 190.

The nozzle 190 forms the narrow plume angle (for example, an angle of 1 to 40 degrees, including endpoints and angles intermittent there between; in one aspect the angle is 5 degrees, 10 degrees, 15 degrees, 20 degrees, 25 degrees, 30 degrees, 35 degrees) multi-stream deposition. The nozzle 190 and resultant angle of the plume produced promotes delivery of the compound to the olfactory region of the user's nasal cavity.

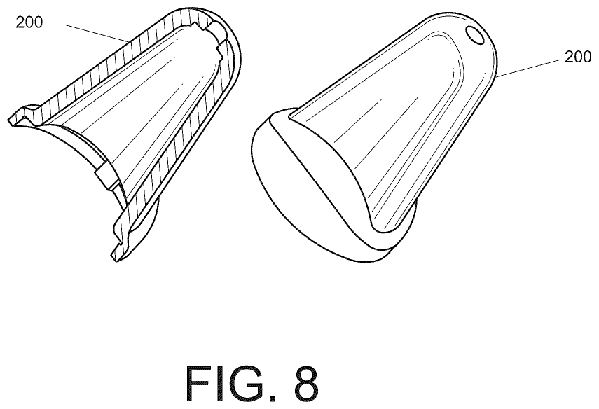

In this implementation, as shown in FIG. 8, the device 100 may include an optional nose cone 200. The external geometries of the nose cone 200 assist in providing proper alignment of the device 100 during insertion into the nose. The diametrically opposed flat sides aid with placement against the septum of either naris, with the depth stop providing correct depth of insertion. The nose cone 200 adds redundancy to nozzle 190 retention through mechanical interference incorporated into the design. As shown in FIG. 3 and FIG. 8, there is an opening in the nose cone 200 which aligns with the nozzle 190. The nose cone 200 is not part of the pressurized flow path.

The housing 110 represents the body of the device 100. The housing 110 includes two different “clamshells” concealing the components of the device 100 and retaining all components to ensure functionality. The housing 110 houses the metered dose pump 130 and pump fitment 180, the actuator grip 210, the y-junction 120, the propellant canister 140, and the dose chamber 150. The nose cone 200 engages onto the outer geometry of the housing 110. The housing 110 is designed to assemble easily through the use of, for example but not limited to, mattel pins, snaps, post or screws, or a combination thereof, molded into the geometry.

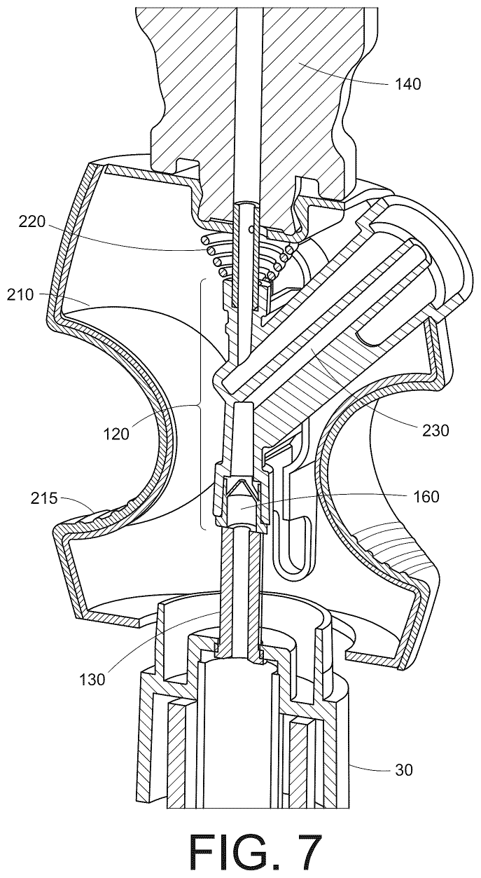

The actuator grip 210 provides for actuation displacement by the user. The actuator grip 210 is composed of two parts, actuator grip A and actuator grip B and surround the y-junction 120 and reside within the housing 110. FIG. 7 shows two finger grip notches 215 are designed into the actuator grip 210 to allow the user to engage the device 100 with the fingers, for example but not limited to, the index and middle finger. These finger grip notches 215 allow the user to apply downward movement leading to device 100 actuation.

The metered dose pump 130 draws compound up from the vial 30 to the y-junction 120. The metered dose pump 130 may utilize a custom pump fitment 180 to promote functionality within the device 100, and allow attachment of the vial 30 via threads. The metered dose pump 130 may deliver, for example but not limited to, volumes, 180 μl, 200 μl, or 230 μl during actuation. Commercially available metered dose pumps 130 can be used.

For the device 100 to consistently deliver compound, the metered dose pump 130 must first deliver compound, followed by propellant canister 140 actuation to expel the compound. As shown in FIG. 7, one manner in which to accomplish this is via a conical spring 220 between the propellant canister 140 and y-junction 120 to create the necessary propellant canister 140 actuation force resulting in the correct order of actuation between the metered dose pump 130 and propellant canister 140. In one implementation, a conical spring 220 is used, although this force is not limited to being produced by a conical spring 220 as other mechanisms can be used. In one aspect, the conical spring 220 has a near zero preload, with a k value of about 25.5 lbf\in and a maximum load of 3.2 Lbf. Selection of the spring or mechanism will include the considerations of: (a) providing for proper device 100 staging; (b) physical space in the device 100; and/or (c) and user feedback regarding how stiff of a conical spring 220 still allows a variety of users to activate the device 100.

The conical spring 220 is installed inline between the propellant canister 140 and y-junction 120. The actuator grip 210 physically holds the propellant canister 140. The user activates the device 100 by, for example, applying an in-line force acting down from the actuator grips 210, and up from the vial 30. This force simultaneously acts to activate both the metered dose pump 130 and the propellant canister 140. The conical spring 220 acts in parallel to the internal propellant canister spring, increasing the necessary force required to activate the propellant canister 140. By choosing the conical spring 220 such that the necessary force required to actuate the propellant canister 140 is in excess of the maximum necessary force required to completely actuate the metered dose pump 130, the device 100 provides that dose is loaded into the dose chamber 150 before propellant gas begins to expel compound from the device 100.

During device 100 actuation, the metered dose pump 130 draws liquid compound up from the vial 30 at the bottom of the device 100 via the y-junction 120, through the internal dose loading channel 230 and into the dose chamber 150. The internal dose loading channel 230 provides a clear route for the compound to be loaded ahead of the annular diffuser 170, without needed to physically pass through the porous material of the annular diffuser 170. As shown in FIG. 6, small arrow heads represent the flow of the propellant while large arrow heads represent the flow of the compound. Priming shots may be required to completely fill the metered dose pump 130 and internal dose loading channel 230 of the y-junction 120 prior to user dosing. A dose cap (not shown) may cover the nose cone 200 of the device 100 and captures the priming shots while also providing a means of visual indication to the user that the device is primed.

In the second stage of device 100 actuation, once the dose chamber 150 has been filled, the propellant canister 140 releases propellant which enters through the top of the y-junction 120, following the path shown by open arrow heads in FIG. 6. The propellant flows physically through the porous material of the annular diffuser 170, which promotions in the vaporization of the propellant. The propellant first contacts the compound at the distal (distal being closer to the nozzle 190, proximal being farther away from the nozzle 190) face of the annular diffuser 150 as seated in the device 100. As the propellant continues to expand, it pushes the compound forward (toward the nozzle 190) in the dose chamber 150, exiting though the nozzle 190 at the end of the dose chamber 150.

The propellant canister 140 provides the propulsive energy for the device 100. The stem of the propellant valve seats into the top receiver of the y-junction 120. During use, the user presses down on the actuator grips 210 which pulls the propellant canister 140 body down, actuating the propellant valve. This releases a metered volume of liquid propellant. As the propellant vaporizes and expands, the compound is forced out of the dose chamber 150 and out through the nozzle 190.

As an example of propellant, but not limited to, the propellant canister 140 uses HFA 134A as the propellant for the system. Other propellants are envisioned. There are commercially available propellant canisters 140.

The device 100, the propellant canister 140, and the vial 30 may all be included or provided together in a kit.

### EXAMPLES AND EMBODIMENTS

### Example 1

The following table provides data on one implementation of the device described herein.

| | Dose Volume [μL] | | | | | | | |
|---|---|---|---|---|---|---|---|---|
| Shot # | Device 1 | Device 2 | Device 3 | Device 4 | Device 5 | Device 6 | | |
| 1 | 190.6 | 193.7 | 185.3 | 199.2 | 199.2 | 145.1 | 185 uL + 10% | 203.5 |
| 2 | 181.4 | 205.5 | 178.9 | 167.7 | 167.7 | 141.7 | 185 uL − 10% | 166.5 |
| 3 | 183.1 | 188.5 | 173.3 | 165.6 | 165.6 | 138.5 | 185 uL + 15% | 212.8 |
| 4 | 183.2 | 193.3 | 145.8 | 164.6 | 164.6 | 136.6 | 185 uL − 15% | 157.3 |
| 5 | 183.3 | 201.5 | 200.7 | 162.0 | 162.0 | 142.1 | | |
| 6 | 185.8 | 207.7 | 166.3 | 179.4 | 179.4 | 138.9 | | |
| 7 | 184.3 | 195.1 | 180.3 | 164.8 | 164.8 | 140.9 | | |
| 8 | 183.3 | 205.4 | 175.3 | 164.9 | 164.9 | 142.0 | | |
| 9 | 180.5 | 178.1 | 172.0 | 164.1 | 164.1 | 141.8 | | |
| 10 | 179.7 | 204.0 | 178.0 | 170.6 | 170.6 | 143.9 | | |
| Mean | 183.5 | 197.3 | 175.6 | 170.3 | 170.3 | 141.2 | | |
| StDev | 3.1 | 9.3 | 14.0 | 11.3 | 11.3 | 2.5 | | |
| Min | 179.7 | 178.1 | 145.8 | 162.0 | 162.0 | 136.6 | | |
| Max | 190.6 | 207.7 | 200.7 | 199.2 | 199.2 | 145.1 | | |

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A device for the intranasal delivery of a compound comprising:
a y-junction including a base, a first branch of the y-junction radiating from the base, a second branch of the y-junction radiating from the base, a third branch of the y-junction radiating from the base, and an internal dose loading channel of the y-junction;
a metered dose pump in fluid communication with the first branch of the y-junction;
a conical spring associated with the second branch of the y-junction;
a dose chamber in fluid communication with the third branch of the y-junction;
a nozzle associated with the dose chamber;
a diffuser between the internal dose loading channel and the dose chamber;
an actuator grip surrounding the y-junction; and
a housing, the y-junction residing within the housing.

Clause 2. The device of any of clauses 1-11, further comprising:
a propellant canister in fluid communication with the second branch of the y-junction and held by the actuator grip, the conical spring between the propellant canister and the second branch of the y-junction.

Clause 3. The device of any of clauses 1-11, further comprising a vial in fluid communication with the metered dose pump.

Clause 4. The device of clause 3, further comprising a pump fitment securing the metered dose pump to the vial.

Clause 5. The device of any of clauses 1-11, further including a check-valve associated between the metered dose pump and the y-junction.

Clause 6. The device of any of clauses 1-11, further including a nose cone in engagement with the housing.

Clause 7. The device of clause 6 further comprising a dose cap covering the nose cone.

Clause 8. The device of any of clauses 1-11, wherein the third branch of the y-junction is at a 45-degree angle from the base of the y-junction.

Clause 9. The device of any of clauses 1-11, wherein the diffuser is annular.

Clause 10. The device of any of clauses 1-11, wherein the diffuser is a porous material.

Clause 11. The device of any of clauses 1-11, wherein the diffuser forms the bottom of the dose chamber.

Clause 12. A device for the intranasal delivery of a compound, the device comprising:
a housing, the housing including a tip, an actuator, and a dose chamber, the tip and the dose chamber in fluid communication within the housing;
a nozzle at a distal portion of the tip, the nozzle providing an outlet for the compound, the nozzle including a plurality of nozzle openings; and
a pump in fluid communication with the dose chamber, the pump to move the compound into the dose chamber upon actuation of the actuator.

Clause 13. The device of any of clauses 12-15, further comprising a propellant canister associated with the housing, the propellant canister having a propellant valve for actuation by the actuator, the propellant canister in fluid communication with the dose chamber.

Clause 14. The device of clauses 12-15, further comprising a vial of compound associated with the pump to move the compound into the dose chamber from the vial.

Clause 15. The device of clauses 12-15, further including a diffuser.

Clause 16. A device for the intranasal delivery of a compound to the olfactory region of the nasal cavity, the device comprising:
a housing, the housing including a tip, an actuator, and a dose chamber, the tip and the dose chamber in fluid communication with the housing;
a nozzle at a distal portion of the tip, the nozzle providing an outlet for the compound, the nozzle including a plurality of nozzle openings;
a pump in fluid communication with the dose chamber, the pump to move the compound into the dose chamber upon actuation of the actuator;
a propellant canister associated with the housing, the propellant canister having a propellant valve for actuation by the actuator, the propellant canister in fluid communication with the dose chamber; and
a vial of compound associated with the pump to move the compound into the dose chamber from the vial wherein the actuator upon actuation of the device compresses the pump moving the compound into the dose chamber and actuation of the propellant valve disperses the propellant pushing the compound providing for the compound to exit the device through the plurality of nozzle openings.

Clause 17. A kit including the device any of clauses 1-16, a propellant canister and a vial.

The present invention is not to be limited in scope by the specific implementations described herein. Indeed, various modifications of the invention in addition to those described herein will become apparent to those skilled in the art from the foregoing description and accompanying figures. Such modifications are intended to fall within the scope of the appended claims.

What is claimed is:

1. A device for the intranasal delivery of a compound comprising:
a y-junction including a base, a first branch of the y-junction radiating from the base, a second branch of the y-junction radiating from the base, a third branch of the y-junction radiating from the base, and an internal dose loading channel of the y-junction;
a metered dose pump in fluid communication with the first branch of the y-junction;
a conical spring associated with the second branch of the y-junction;
a dose chamber in fluid communication with the third branch of the y-junction;
a nozzle associated with the dose chamber;
a diffuser between the internal dose loading channel and the dose chamber;
an actuator grip surrounding the y-junction; and
a housing, the y-junction residing within the housing.
